(19) United States
(12) Patent Application Publication
(10) Pub. No.: US 2018/0141911 A1
(43) Pub. Date: May 24, 2018

(54) Quinoline Derivatives for Diagnosis and Treatment of Alzheimer's Disease

(71) Applicant: Northeastern University, Boston, MA (US)

(72) Inventor: Robert N. HANSON, Newton, MA (US)

(21) Appl. No.: 15/572,999

(22) PCT Filed: May 16, 2016
(86) PCT No.: PCT/US2016/032700

§ 371 Date: Nov. 9, 2017

Related U.S. Application Data
(60) Provisional application No. 62/161,473, filed on May 14, 2015.

(51) Int. Cl. C07D 215/40; C07D 215/24; C07D 401/06; A61K 51/04; A61P 25/28

(52) U.S. Cl. CPC C07D 215/40; C07D 215/24; A61P 25/28; A61K 51/0455; C07D 401/06

(57) ABSTRACT

A new class of quinoline compounds is useful for the detection and treatment of Alzheimer's disease and other neurodegenerative diseases such as amyloidoses and tauopathies. The compounds can be synthesized in radiolabeled form for use as imaging agents, which can be used for early detection of aggregates in the brain or other tissues prior to onset of symptoms, allowing early therapeutic intervention. The compounds are also useful for the prevention and treatment of such diseases.

### BACKGROUND

Alzheimer's disease (AD) is a neurodegenerative disease characterized by progressive loss of cognition and motor control. Although symptoms of AD generally arise later in life, clinical diagnosis often occurs at a much earlier stage. While pharmacological intervention may delay the cognitive decline, the disease is progressive, with no known cure, and eventually death ensues. The emotional and financial burdens borne by the individuals and their care givers are high. Cost of care and treatment are estimated to rise to more than $1 trillion annually by 2050, which has generated considerable interest in developing improved methods for earlier diagnosis and more effective intervention (1).

Although diagnosis of AD is still based upon clinical evaluation of patients, post-mortem evaluation of the brains of patients has provided a number of potential biomarkers for non-invasive detection. In particular, the presence of increased levels of amyloid beta (Aβ) plaques and tau neurofibrillary tangles (NFT) has given rise to competing hypotheses concerning the genesis of the disease (2). In most cases, both features are present, although they may not progress at the same rate. One result of these conflicting findings is the development of divergent non-invasive imaging strategies. Most efforts at developing an imaging agent for AD have focused on agents having an avidity for Aβ, largely as a result of modifications of the thioflavin-T dye, which is used to stain plaques in post-mortem brain (3). This observation gave rise to [¹¹C]-PiB, the first clinically useful AD imaging agent (4,5), and subsequently to [¹⁸F]-fluorbetapir (6,7), [¹⁸F]-florbetaben (8,9) and [¹⁸F]-flutemetamol (10,11), all approved by the FDA (see FIG. 1A). The development of these agents, as well as many other analogs and derivatives, has been the subject of recent reviews (12-17). FIG. 1B illustrates a number of potential imaging agents, which are either in clinical trials or in preclinical studies. These all possess two aromatic rings linked through a linking group, and a radionuclide is either part of the central core or appended as a prosthetic group on the periphery. All of these agents show a preference for binding Aβ plaque as opposed to tau NFTs. The structure-localization relationships for NFT (tau)-avid imaging agents were the subject of number of recent articles (18-24); clearly they are divergent from the Aβ-selective agents (see FIG. 1C).

Metal chelating agents that bind to aggregates of tau and Aβ have been investigated. These agents are characterized by avidity for metal ions, including ions of iron, copper and zinc. 8-Hydroxyquinoline and its halogenated derivative clioquinol, known chelators of these metal ions, have been shown to bind to both tau and Aβ aggregates in post-mortem AD brain tissues (25-29). Radiochemical methods were developed for the rapid preparation of either radioiodinated or radiofluorinated products, and the compounds were evaluated in AD mouse models or as imaging agents (22,25,30-32). Of particular interest were the [¹⁸F]-2-fluoro-8-hydroxyquinolines, which displayed significant binding to the Aβ-protein in vivo in rodent models and were taken on to nonhuman primates. Unfortunately, [¹⁸F]-2-fluoro-8-hydroxyquinoline ([¹⁸F]-CABS13) demonstrated poor brain penetration in the normal subjects, and was accompanied by rapid, high conversion to a polar metabolite (25). For therapy, chelating groups that sequester copper and iron ions tend to inhibit aggregation of β-amyloid and tau proteins (26-28). Efforts to couple these properties together have generated hybrid compounds that exhibit high affinity for the Aβ and tau proteins and sequester metal ions (33,34) Two examples of such hybrid compounds are shown in FIG. 1D.

Both Aβ-selective and tau-selective approaches appear to be capable of discriminating between AD and non-AD patients when the disease burden is sufficiently high and symptoms are advanced. However, there remains an urgent need for diagnostic agents that can detect the nascent disease, i.e., before clinical symptoms are apparent and at a point when pharmacological intervention can delay or block the progression of the disease. The availability of such agents also would facilitate the development of such pharmacological agents by allowing non-invasive monitoring of disease models in non-human species.

### SUMMARY OF THE INVENTION

The invention provides a new class of quinoline compounds for use in the detection and treatment of Alzheimer's disease. The compounds recognize amyloid-beta and/or tau proteins associated with Alzheimer's disease and other neurodegenerative diseases such as amyloidoses and tauopathies. The compounds can be synthesized in radiolabeled forms for use as imaging agents, which can be used for early detection of aggregates in the brain or other tissues prior to onset of symptoms, allowing early therapeutic intervention.

One aspect of the invention is a compound having the formula:

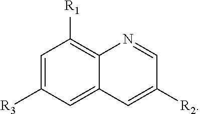

R₁ is OH, OCH₂C₆H₅, NO₂, NH₂, aminoacyl, aminosulfonyl, NHCH₃, or N(CH₃)₂; and R₂ and R₃ are independently F, Cl, Br, I, or aryl, heteroaryl, CH═CH-aryl, or CH═CH-heteroaryl, wherein any of the aforementioned aryl groups can be halo-substituted at any position. In certain embodiments the compound is used as an imaging agent for amyloid plaques and/or neurofibrillary tangles, such as amyloid plaques containing beta amyloid (Aβ) neurofibrillary tangles containing tau, and one or both of R and R₃ include a radioisotope of F, Br, or I, such as ¹⁸F or ¹²³I. 4. In certain embodiments, the compound prevents and/or reverses the formation of amyloid plaques and/or neurofibrillary tangles in a mammal, such as in the brain of the mammal. The mammal can be a human. In certain embodiments, the compound binds a metal ion, such as an ion of iron, copper, or zinc. In certain embodiments, the compound is an active component of a pharmaceutical composition.

Another aspect of the invention is a method of diagnosing or prognosing a disease or condition in a subject associated with the presence of amyloid deposits and/or neurofibrillary tangles in an organ or tissue of the subject. The method includes the step of administering a compound described above to the subject. The administered compound binds to and aids in the detection of amyloid deposits and/or neurofibrillary tangles. The subject can be a mammal, such as a human.

Yet another aspect of the invention is a method to aid in treating or preventing a disease or condition in a subject associated with a presence of amyloid deposits and/or neurofibrillary tangles. The method includes the step of administering a compound described above to the subject. The administered compound decreases the amount of amyloid deposits and/or neurofibrillary tangles in the subject. The subject can be a mammal, such as a human.

Still another aspect of the invention is a method of synthesizing a compound described above. The method includes the steps of: (a) preparing an intermediate which is 8-(benzyloxy)-3-(tributylstannyl)quinoline or 8-(benzyloxy)-6-(tributylstannyl)quinoline; (b) subjecting the intermediate to halodestannylation to obtain 8-(benzyloxy)-3-haloquinoline or 8-(benzyloxy)-6-haloquinoline; and (c) subjecting the product obtained in (b) to debenzylation to obtain 3-halo-8-hydroxyquinoline or 6-halo-8-hydroxyquinoline. Halo represents F, Br, or I, and in methods for the preparation of an imaging agent, the halodestannylation is radiohalodestannylation, and 3-radiohalo-8-hydroxyquinoline or 6-radiohalo-8-hydroxyquinoline is obtained, wherein halo represents a radioisotope of F, Br, or I, such as ¹⁸F or ¹²³I.

The invention can be further summarized with the following listing of embodiments:
1. A compound having the formula:

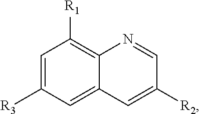

wherein R₁ is selected from the group consisting of OH, OCH₂C₆H₅, NO₂, NH₂, aminoacyl, aminosulfonyl, NHCH₃, and N(CH₃)₂; and R₂ and R₃ are independently from the group consisting of F, Cl, Br, I, or aryl, heteroaryl, CH═CH-aryl, or CH═CH-heteroaryl, wherein any of the aforementioned aryl groups can be halo-substituted at any position.
2. The compound of embodiment 1, wherein at least one of R and R₃ is selected from radioisotopes of F, Br, and I.
3. The compound of embodiment 2, wherein at least one of R₂ and R₃ is ¹⁸F or ¹²³I.
4. The compound of any of the preceding embodiments that binds to amyloid plaques and/or neurofibrillary tangles in a mammal.
5. The compound of embodiment 4, wherein the amyloid plaques comprise beta amyloid (Aβ) and/or the neurofibrillary tangles comprise tau.
6. The compound of any of the preceding embodiments that prevents and/or reverses the formation of amyloid plaques and/or neurofibrillary tangles in a mammal.
7. The compound of any of the preceding embodiments that binds a metal ion.
8. The compound of embodiment 7, wherein the metal ion is an ion of iron, copper, or zinc.
9. The compound of embodiment 1 that is selected from the following compounds, wherein X is a halogen selected from F, Br, and I, and wherein R is H or C1-C6 alkyl:

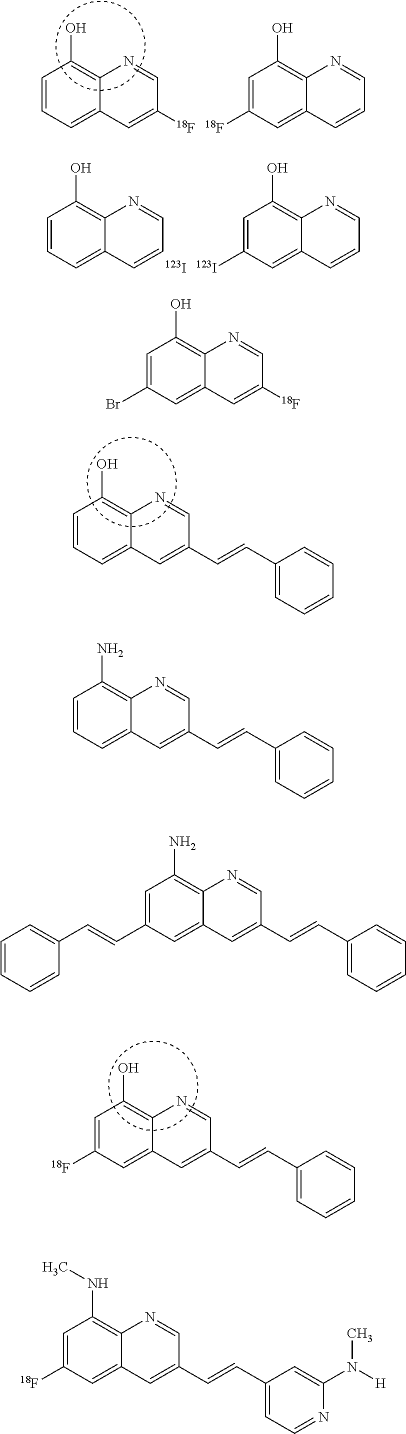

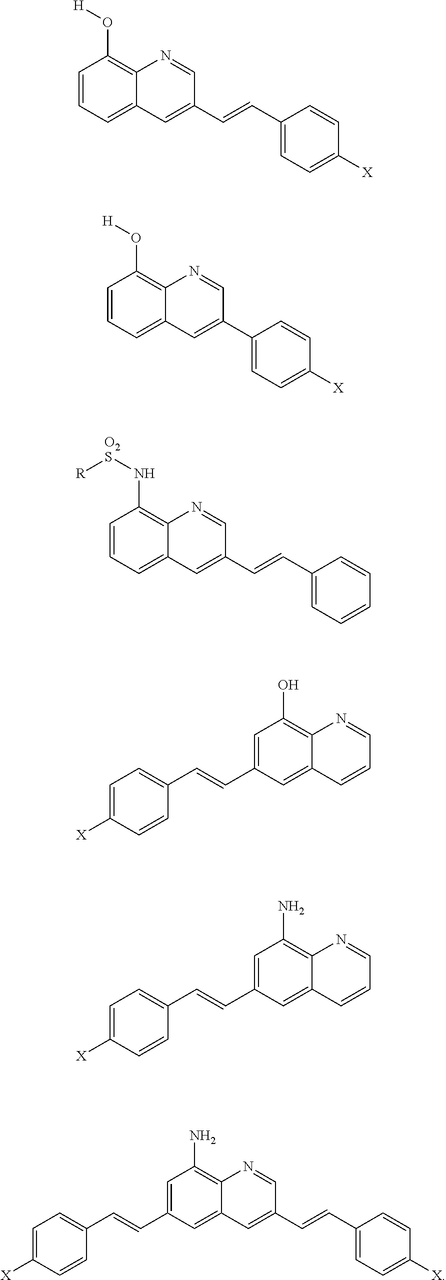

10. The compound of any of the preceding embodiments that is capable of crossing the blood-brain barrier of a mammal.
11. The compound of any of the preceding embodiments that is an imaging agent for visualizing amyloid beta plaques and/or tau neurofibrillary tangles in the brain of a mammal.
12. A pharmaceutical composition comprising the compound of any of the preceding embodiment and an excipient, carrier, or diluent.
13. A method of diagnosing or prognosing a disease or condition in a subject, the disease or condition associated with a presence of amyloid deposits and/or neurofibrillary tangles in an organ or tissue of the subject, the method comprising the step of administering a compound of any of the preceding embodiments to the subject, whereby the presence of amyloid deposits and/or neurofibrillary tangles is detected.
14. The method of embodiment 13, wherein the disease or condition is associated with the presence of amyloid deposits, and wherein the disease or condition is selected from the group consisting of Alzheimer's disease, Parkinson's disease, Huntington's disease, spongiform encephalopathy, diabetes mellitus type 2, fatal familial insomnia, atrial amyloidosis, atherosclerosis, rheumatoid arthritis, familial amyloid polyneuropathy, hereditary non-neuropathic systemic amyloidosis, dialysis related amyloidosis, cerebral amyloid angiopathy, and systemic AL amyloidosis.
15. The method of embodiment 13, wherein the disease or condition is associated with the presence of neurofibrillary tangles, and wherein the disease or condition is selected from the group consisting of Alzheimer's disease, progressive supranuclear palsy, chronic traumatic encephalopathy, primary age-related tauopathy, corticobasal degeneration, and postencephalitic parkinsonism.
16. A method to aid in treating or preventing a disease or condition in a subject, the disease or condition associated with a presence of amyloid deposits and/or neurofibrillary tangles in the subject, the method comprising the step of administering a compound of any of embodiments 1-11 or the pharmaceutical composition of embodiment 12 to the subject, whereby amyloid deposits and/or neurofibrillary tangles in the subject are decreased.
17. The method of embodiment 16, wherein the disease or condition is associated with the presence of amyloid deposits, and wherein the disease or condition is selected from the group consisting of Alzheimer's disease, Parkinson's disease, Huntington's disease, spongiform encephalopathy, diabetes mellitus type 2, fatal familial insomnia, atrial amyloidosis, atherosclerosis, rheumatoid arthritis, familial amyloid polyneuropathy, hereditary non-neuropathic systemic amyloidosis, dialysis related amyloidosis, cerebral amyloid angiopathy, and systemic AL amyloidosis.
18. The method of embodiment 16, wherein the disease or condition is associated with the presence of neurofibrillary tangles, and wherein the disease or condition is selected from the group consisting of Alzheimer's disease, progressive supranuclear palsy, chronic traumatic encephalopathy, primary age-related tauopathy, corticobasal degeneration, and postencephalitic parkinsonism.
19. A method of synthesizing a compound of any of embodiments 1-11, the method comprising the steps of:

(a) preparing an intermediate compound which is 8-(benzyloxy)-3-(tributylstannyl)quinoline or 8-(benzyloxy)-6-(tributylstannyl)quinoline;

(b) subjecting the intermediate to halodestannylation to obtain 8-(benzyloxy)-3-haloquinoline or 8-(benzyloxy)-6-haloquinoline; and

(c) subjecting the product obtained in (b) to debenzylation to obtain 3-halo-8-hydroxyquinoline or 6-halo-8-hydroxyquinoline; wherein halo represents F, Br, or I.
20. The method of embodiment 19, wherein step (b) comprises radiohalodestannylation, and in step (c) 3-radiohalo-8-hydroxyquinoline or 6-radiohalo-8-hydroxyquinoline is obtained.

### BRIEF DESCRIPTION OF THE DRAWINGS

FIG. 1A shows several prior art therapeutic and diagnostic agents directed to binding amyloid beta (Aβ). FIG. 1B shows a number of prior art potential imaging agents which are in clinical trials or in preclinical studies. FIG. 10 shows several prior art tau-selective imaging agents. FIG. 1D shows prior art hybrid metal-binding Aβ/tau protein avid therapeutic agents for inhibition of protein aggregation.

FIGS. 2A and 2B show compounds of the invention based on a 3-,6-substituted-8-(hydroxyl/amino)quinolone structure.

FIG. 3 shows a scheme for the radiosynthesis of 3-radiohalogenated-8-hydroxyquinolines.

FIG. 4 shows a scheme for the preparation of 3-iodo- and 3-fluoro-8-hydroxyquinoline via methods appropriate for radiosynthesis. Reagents and conditions: (i) NBS, HOAc; (ii) Fe. HOAc—H₂O; 70% H₂SO₄, 220° C., 72 h, (iv) C₆H₅CH₂Br, K₂CO₃, DMF; (v) Bu₆Sn₂, Pd(0), (vi) NIS, dioxane; (vii) Selectfluor, AfOTf; (viii) BBr₃, (CH₃)₅C₆H.

FIGS. 5A-5D show additional synthetic schemes to produce compounds of the invention.

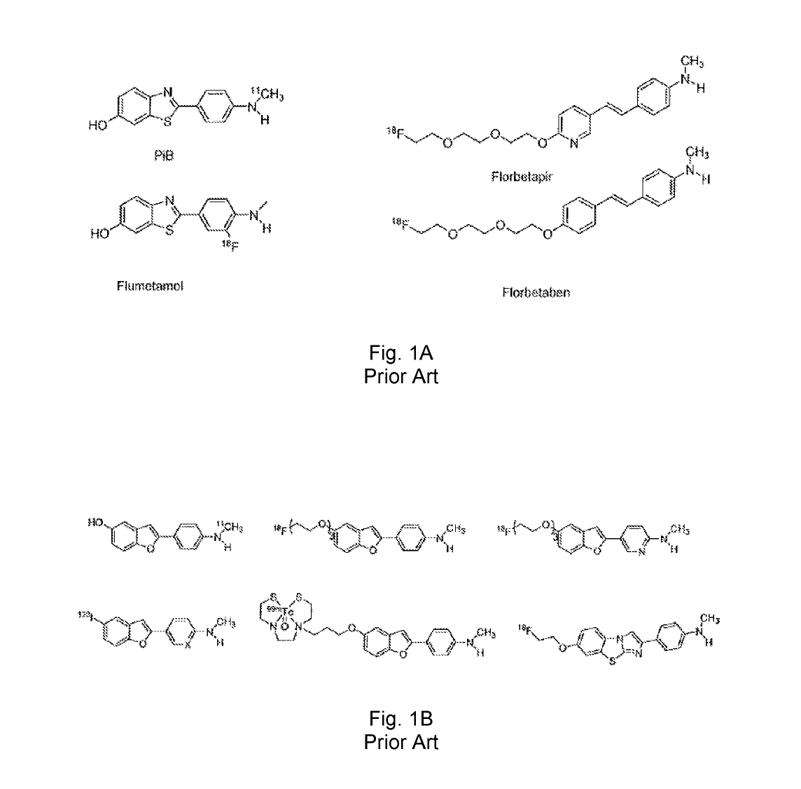

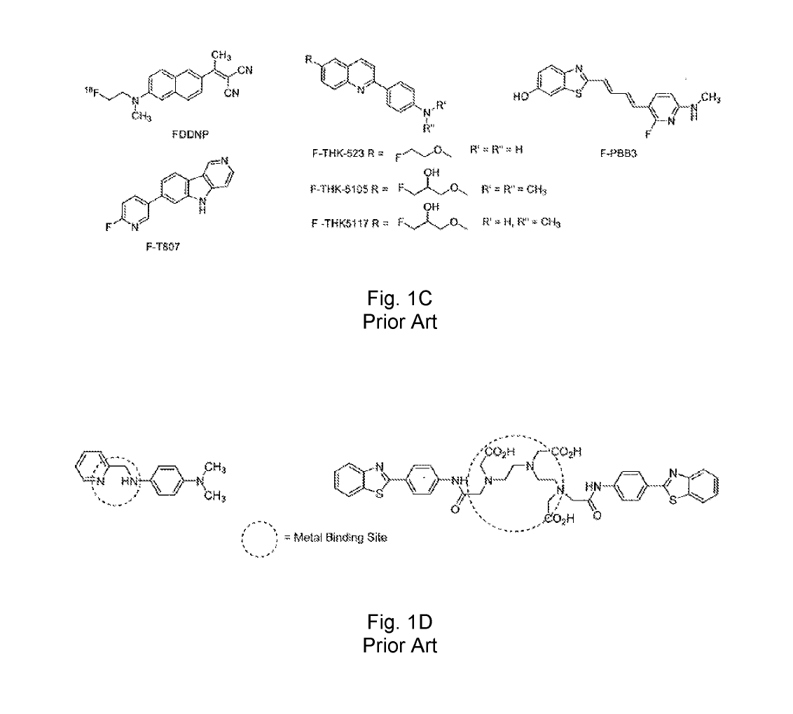

### DETAILED DESCRIPTION OF THE INVENTION

The invention provides a new class of quinoline compounds for use in the detection and treatment of Alzheimer's disease and other neurodegenerative diseases related to the accumulation of amyloid or neurofibrillary tangles. The invention also provides novel approaches to the synthesis of both unlabeled and labeled forms of the quinoline compounds. The molecular structure of the compounds includes features that recognize amyloid-beta and/or tau proteins associated with Alzheimer's disease and also bind metal ions such as Cu²⁺ and Fe²⁺ associated with the disease process. The compounds can inhibit the aggregation process that leads to the characteristic plaques and tangles and prevent the development and progression of Alzheimer's disease. In their radiolabeled forms (e.g., ¹⁸F or ¹²³I), these compounds can non-invasively detect aggregates in the brain prior to or following the onset of symptoms, allowing specific therapies to be initiated so as to retard, stop, or reverse the disease process.

The compounds of the invention are quinoline derivatives substituted at the 8 position with a substituent that, together with the nitrogen of the quinoline ring, preferably can form a coordination structure with a metal ion, such as metal ions that promote or are associated with the formation of amyloid plaque and/or neurofibrillary tangles. The compounds are also substituted at the 3 and/or 6 positions with substituents containing a halide for use as a radiolabel in imaging applications. These substitution sites are chemically and metabolically stable and have little effect on the chelating capacity of the quinoline moiety. Each of the substitution sites can be individually manipulated to facilitate optimization of chemical and biological properties. The compounds can also have antibacterial and/or antifungal activity.

Prior efforts to prepare labeled 8-hydroxyquinoline derivatives have used substituents at positions either ortho or para (i.e., 5-,7-) to the 8-hydroxy group. Labels were introduced via electrophilic substitution, or ortho/para (2-,4-) to the 1-nitrogen, which can be introduced from the corresponding-one derivative. In all of these cases, the properties of the chelating moiety (8-OH and 1-N) are significantly affected by resonance. The 2-fluoro derivative, in addition to reducing the electron density at 1-N, is also susceptible to nucleophilic displacement. These derivatives were prepared and evaluated in the prior art largely because they were synthetically accessible and not necessarily because they would have the optimal properties.

The 3-position of the quinoline ring, however, has distinct advantages, as it only exerts a modest inductive effect on either the 8-OH or the 1-N chelating group. It also is stable against displacement by endogenous nucleophiles. A review of the literature indicates that very few 3-halogenated 8-hydroxy quinolines have been reported, largely because of the difficulties associated with their synthesis. Nevertheless, because radiotracers require relatively small quantities for undertaking imaging studies, the key steps are the final radiolabeling and purification step, and low yields in the initial portion of the synthesis can be tolerated.

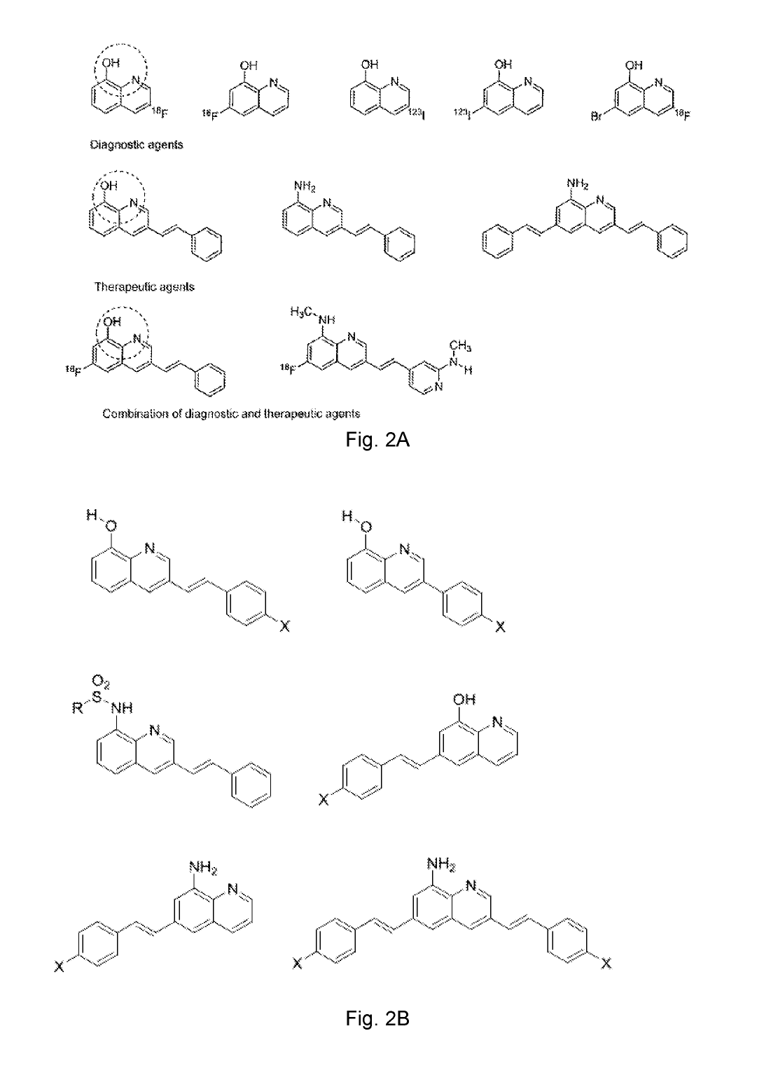

The present invention combines the metal binding capacity of 8-hydroxy/amino quinolines, with the Aβ/tau protein seeking capacity of the styryl moiety to generate a new class of hybrid agents (see examples shown in FIGS. 2A-2B) for detecting and/or treating Aβ/tau aggregates in humans and other mammals. These compounds can be prepared via a novel scheme starting from commercially available 8-nitroquinoline. The presence of the nitro group results in the introduction of functional groups selectively at the 3- and 6-positions. These sites are not accessed by electrophilic substitution on 8-hydroxy or 8-aminoquinolines which generate 5- and 7-substitution. Skraup synthesis gives 6- but not 3-substituted quinolines. Other syntheses via anilines generate 2- and 4-substituted 8-hydroxy/amino quinolines, but not the 3-substituted quinolines. The process of the invention can interconvert the initial 3-/6-bromo intermediates via stannyl or boronyl reagents to radiohalogenated products for imaging or to the aryl(heteroaryl)/vinylaryl(heteroaryl) products for therapy. The process is efficient and versatile. One can replace the 8-hydroxy group with alkoxy groups or generate the 8-amino analog which can be converted to mono/di-substituted alkyl/acyl derivatives.

The invention includes 3-,6-substituted-8-(hydroxyl/amino)quinoline compounds described by Formula (I) below.

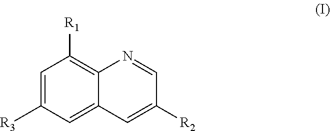

In compounds of Formula (I), R₁ can be OH, OCH₂C₆H₅, NO₂, NH₂, aminoacyl, aminosulfonyl, NHCH₃, or N(CH₃)₂; and R₂ and R₃ can be independently F, Cl, Br, I, or aryl, heteroaryl, CH═CH-aryl, or CH═CH-heteroaryl, wherein any of the aforementioned aryl groups can be halo-substituted at any position. For use as radiodiagnostics, compounds of Formula (I) can include any radioisotopes of F, Br, and I. A number of specific compounds of Formula (I) suitable for use as therapeutic or diagnostic agents are shown in FIGS. 2A-2B.

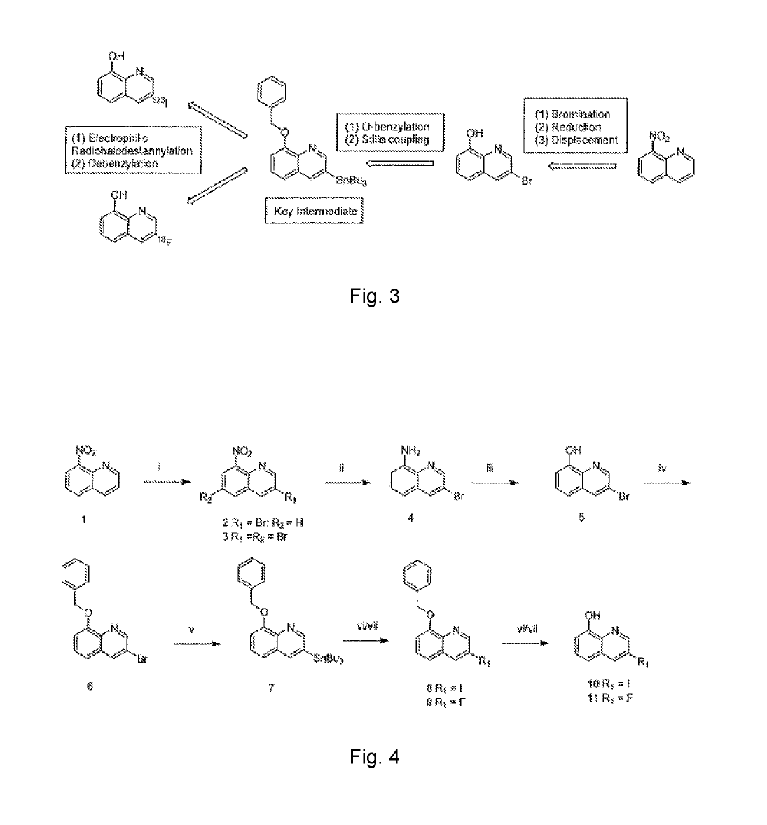

Precursors were prepared that can be converted to either radioiodinated derivatives for SPECT or radiofluorinated derivatives for PET. The approach is shown in FIG. 3. It should be noted, that the previously reported syntheses of 3-iodo and 3-fluoro-8-hydroxyquinoline are not appropriate for translation to the radiolabeled materials. In the synthetic approach of the invention, however, the radiohalogenated products can be derived from a tri-n-butylstannylated intermediate using an electrophilic radiofluoro/iododestannylation method. This key stannylated intermediate can be prepared using 3-bromo-8-benzyloxyquinoline, the protected derivative of 3-bromo-8-hydroxyquinoline. 3-Bromo-8-hydroxyquinoline was not commercially available, and a process was developed by which sufficient quantities of the requisite precursor could be obtained to undertake radiochemical studies.

The synthesis of the precursor (see FIG. 4) began with the bromination of 8-nitroquinoline 1 using N-bromosuccinimide. The presence of the nitro group reverses the reactivity of the aromatic rings, with the 3-position constituting the most electron rich site. As the reaction proceeds and the starting material is depleted, bromination at the 6-position of the 3-brominated product 2 also becomes a competing reaction. Use of excess NBS can essentially consume all of the starting material but at the expense of the mono-brominated product. Therefore, only a slight excess of reagent was employed and a chromatographic separation gave the pure product 2 distinct from the dibrominated 3 and nonbrominated 1 materials. The 8-nitro group was reduced using iron powder and acetic acid to give the 3-bromo-8-aminoquinoline 4. Recrystallization gave the pure product needed for the subsequent displacement reaction. Previous syntheses of the 3-bromo-8-hydroxyquinoline reported good yields for the nucleophilic substitution product from 8-aminoquinoline. Conditions were 70% sulfuric acid at 220-230° C. in a sealed tube, however, in our hands this was a more complex reaction. The reaction appeared to be sensitive to both the concentration of sulfuric acid and temperature. Use of lower concentrations (50-60%) at 220-230° C. gave only the 8-hydroxy product 5, albeit in low yields (10-25%) and the reaction required 5-7 days. Higher temperatures resulted in a complex mixture of materials, including significant decomposition. Higher concentrations of acid (>75%) also caused decomposition and lower yields (<15%) over a range of temperatures (220-250° C.) and times (1-5 days). Ultimately, the conditions that were most consistent for this conversion to 5 were 65% sulfuric acid, 230° C. for 2-4 days, which gave yields in the 30-40% range.

Protection of the 8-hydroxy group was achieved by benzylation with benzyl bromide in DMF to give 6 in good yields. Based upon the studies with the 2-fluoro analog, this group could eventually be removed by hydrogenation or acid (22). Stannylation with hexabutyl ditin and Pd(0) gave the tri-n-butylstannyl quinoline derivative 7 in good overall yields and in high purity. The stannylated intermediate was rapidly converted directly to the corresponding 3-iodo-8-benzyloxyquinoline 8 using N-iodosuccinimide (NIS) in good overall yields. Although hydrogenation had been used previously to deprotect the 2-fluoro analog, conditions were avoided here that may cause reductive dehalogenation, especially for the 3-iodo product. The 3-bromo-8-benzyloxyquinoline 6 was evaluated with trifluoroacetic acid in dichloromethane, conditions which reportedly remove benzyl groups quickly. In the hands of the inventor, however, debenzylation was a slow process. Rather than exploring alternative protecting groups, other conditions were examined. Boron tribromide is more often used for removing O-methyl groups; however, the inventor considered that the presence of the adjacent N1-group could activate the Lewis acid and facilitate the process. Indeed, BBr₃ regenerated 3-bromo-8-hydroxyquinoline 5 in less than 15 minutes at ambient temperature. Application of these conditions to the 3-iodo intermediate resulted in a rapid and complete conversion to 3-iodo-8-hydroxyquinoline 10. Therefore, demonstration of a practical route to the radioiodinated derivative was established. Fluorodestannylation was achieved using SELECTIFLUOR and silver triflate in dichloromethane. The reaction proceeded rapidly to give the 3-fluoro-8-benzyloxyquinoline 9 which underwent facile debenzylation with BBr₃ to give 3-fluoro-8-hydroxyquinoline 11 in good isolated yields.

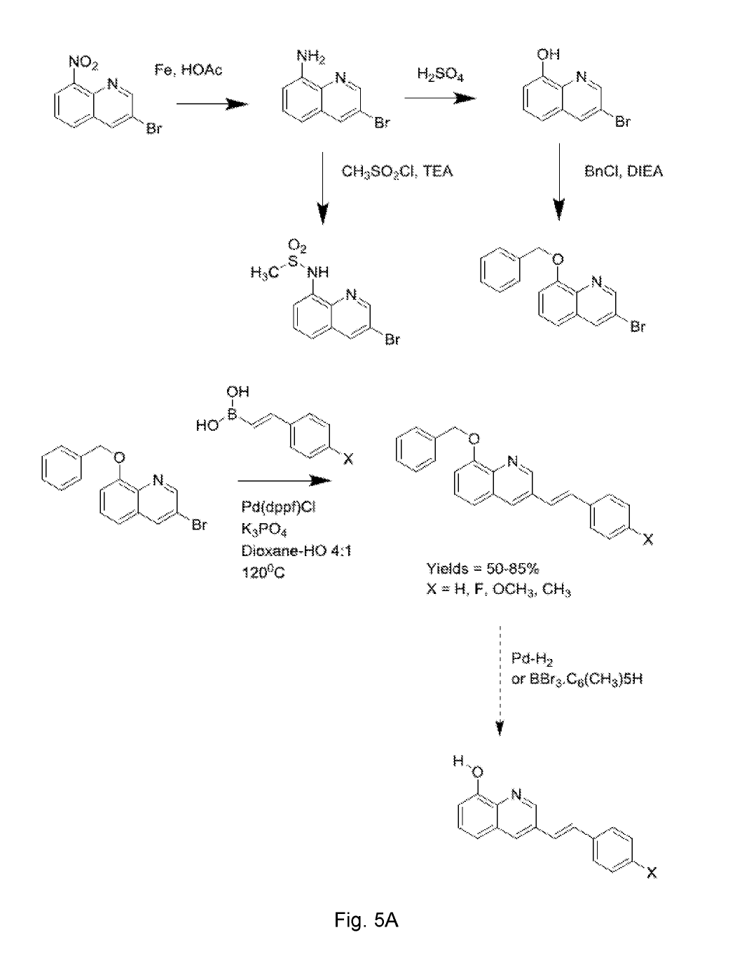

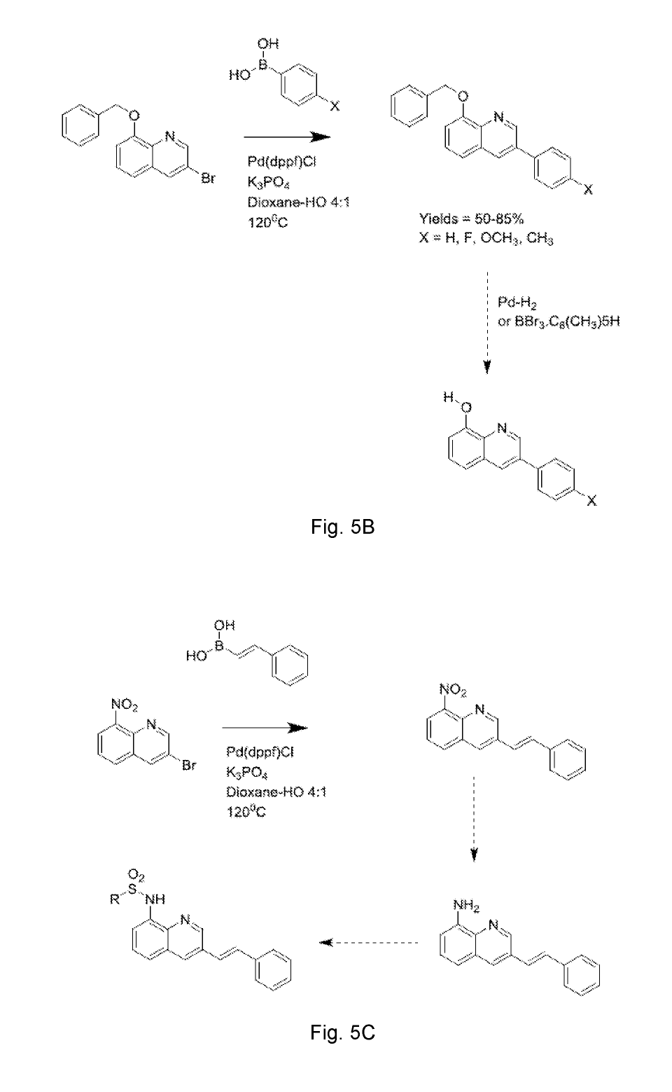

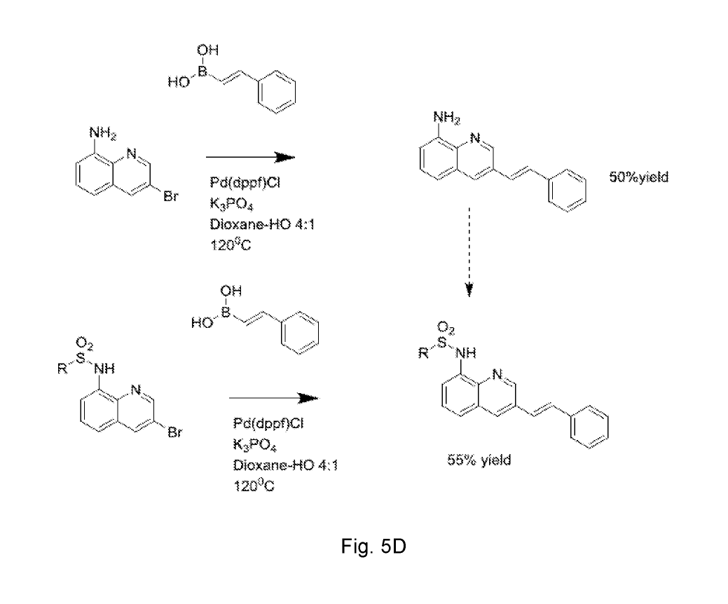

FIGS. 5A-5D illustrate additional synthesis pathways that can be used to produce compounds of the invention.

In summary, novel imaging agents based on the 8-hydroxyquinoline scaffold have been demonstrated. An efficient synthetic scheme for the radiosynthesis of either ¹²³I- or ¹⁸F-radiotracers has been developed using 3-tri-n-butylstannyl-8-benzyloxyquinoline as the key intermediate (precursor). The final two steps proceed rapidly and in high yield and can be translated directly to a radiochemical process.

### EXAMPLES

### Example 1

### Reagents and Instrumentation

All reagents were purchased and used as received, without purification. Silica gel chromatography was performed on 35-70 mesh silica gel (60 Å) using glass columns. All ¹H NMR was obtained on a Varian spectrometer (400 MHz) at room temperature. Reported chemical shifts (δ) are given in parts per million (ppm) and are referenced to chloroform-d (δ=7.26) or tetramethylsilane (δ=0.00). Melting points were determined in a Thermo Scientific manual melting point apparatus. LC-MS analysis was conducted on a HPLC SUNFIRE C18 column (4.6 mm×50 mm, 3.5 μm column; 10 μL injection; 30% to 100% acetonitrile/water and 0.1% formic acid gradient; 150 mL/min flow rate) with 254 nm UV single wave length detection and Waters Micromass ZQ time-of-flight (TOF) mass spectrometer (electrospray ionization). Analytical thin-layer chromatography (TLC) was performed on polyester sheets pre-coated with silica gel matrix 60 F254 obtained from Sigma-Aldrich and visualized under UV light or stained with iodine.

### Example 2

### Synthesis of 3-Bromo-8-nitroquinoline

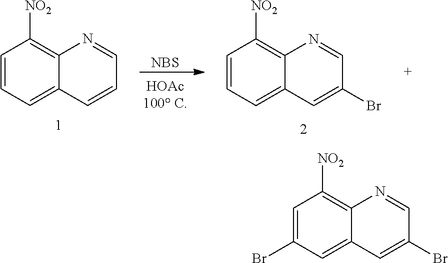

To 5 mL of acetic acid were added 8-nitroquinoline (0.2 g, 1.15 mmol) and N-bromosuccinimide (0.102 g, 0.575 mmol). The solution was heated to reflux for 2 h, cooled to ambient temperature, and poured over 20 mL of water. The crude product was collected by filtration, washed with cold water and air dried. The crude product (a mixture of monobrominated, dibrominated, and unbrominated material) was purified by column chromatography (9 g) using 3:1 hexane/ethyl acetate as the eluent. Chromatography gave 2 as a white solid (0.121 g, 42% yield, mp 122° C., lit mp 123° C.) as well as the 3,6-dibromo-8-nitroquinoline (0.054 g, mp: 179° C.). ¹H NMR (400 MHz, CDCl₃) δ 9.05 (1H, s), 8.43 (1H, s), 8.05-8.07 (1H, d, J=7.2 Hz), 7.97 (1H, d, J=8.8 Hz), 7.7 (1H, t, J=8.0 Hz) for 2. ¹H NMR (400 MHz, CDCl₃) δ 9.04 (1H, d, J=1.2 Hz), 8.34 (1H, d, J=2.4 Hz), 8.12 (2H, d, J=1.2 Hz) for 3,6-dibromo-8-nitroquinoline.

### Example 3

### Synthesis of 3-Bromoquinolin-8-amine

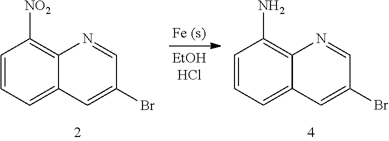 [SMILES: O=[N+]([O-])c1cccc2cc(Br)cnc12 ; Nc1cccc2cc(Br)cnc12]

Intermediate 2 (0.847 g, 3.3 mmol) was dissolved in 30 mL of acetic acid-water (2:1), followed by the addition of iron powder (0.935 g, 16.7 mmol). The reaction mixture was stirred at ambient temperature for 18 hours. Solvent was removed by rotary evaporation; the product mixture was suspended in dichloromethane and filtered through CELITE to remove iron salts. The resultant solid was purified by column chromatography on silica gel (35 g) using dichloromethane as the eluent. Fractions containing the product were combined, evaporated to dryness to yield 4 as a yellow solid 0.45 g (60% yield, mp 104° C., lit mp=106-107° C.). ¹H NMR (400 MHz, CDCl₃) δ 8.72 (1H, d, J=2.4 Hz), 8.21 (1H, d, J=2.0 Hz), 7.32-7.38 (1H, t, J=8.0 Hz), 7.06 (1H, d, J=8.0 Hz), 6.92 (1H, d, J=7.2 Hz), 4.8-5.2 (3H, s).

### Example 4

### Synthesis of 8-Hydroxy-3-bromoquinoline

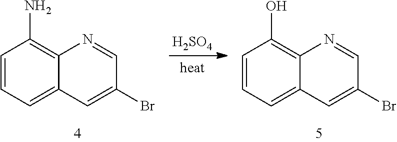 [SMILES: Nc1cccc2cc(Br)cnc12 ; "H2SO4" ; Oc1cccc2cc(Br)cnc12]

8-Amino-3-bromoquinoline (4) (0.3 g, 1.3 mmol) was dissolved in 10 mL of 70% sulfuric acid and placed in a Q-Tube (Sigma Aldrich). The tube was sealed and heated with stirring at 220° C. for 3 days. After cooling to ambient temperature, the reaction solution was brought to pH 8 by addition of NH₄OH (16 M), followed by extraction with ethyl acetate (4×25 mL). The organic phases were combined, dried over Na₂SO₄, filtered, and evaporated to dryness. The crude product was purified by column chromatography on silica gel (7 g) using 3:1 dichloromethane/hexane as the eluent. Fractions containing the product were combined and evaporated to dryness to give 5 as a white solid 0.184 g (34% yield, mp 99-101° C., lit mp 111.5-112.5° C.). ¹H NMR (400 MHz, CDCl₃) δ 8.77 (1H, d, J=2.0 Hz), 8.32 (1H, d, J=2.0 Hz), 7.99 (1H, s), 7.48 (1H, t, J=8.0 Hz), 7.18 (1H, dd, J=23.2 Hz, J=9.6 Hz). LCMS found 225.90, [M+2H]⁺.

### Example 5

### Synthesis of 8-(Benzyloxy)-3-bromoquinoline

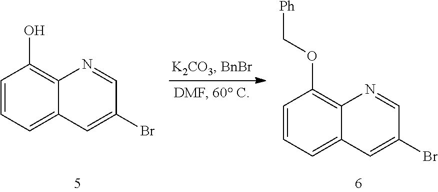

3-Bromo-8-hydroxyquinoline 5 (0.105 g, 0.48 mmol) was dissolved in 3 mL of anhydrous DMF containing K₂CO₃ (0.071 g, 0.515 mmol), followed by addition of benzylbromide (0.088 g, 0.515 mmol). The reaction mixture was stirred at 60° C. for 2 h. The reaction mixture was poured over water and extracted with ethyl acetate (3×25 mL). The combined organic phases were washed with brine; then dried over Na₂SO₄ (anhydrous). The mixture was filtered, evaporated to dryness, and the crude product was purified by column chromatography on silica gel (6 g) using a 1:1 dichloromethane/hexane to 100% dichloromethane gradient. fractions containing product were combined, followed by removal of solvent, to give 6 as a pale yellow solid (0.114g, 77.5% yield, mp 120° C.). ¹H NMR (400 MHz, CDCl₃) δ 8.94 (1H, d, J=2.4), 8.28 (1H, d, J=2.0 Hz), 7.49 (2H, d, J=7.2 Hz), 7.27-7.4 (5H, m), 7.03 (1H, d, J=8.0 Hz), 5.41 (2H, s). MP: 120° C. LCMS found 313.89, [M+H]⁺.

### Example 6

### Synthesis of 8-(Benzyloxy)-3-(tributylstannyl)quinoline

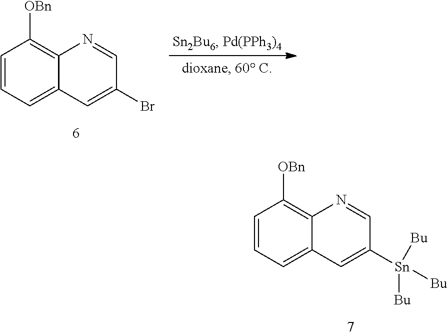

8-(Benzyloxy)-3-bromoquinoline (6) (0.250 g, 0.796 mmol) was added to 8 mL of dry dioxane containing 5% mole equivalents of tetrakis(triphenylphosphine)palladium(0) (0.046 g, 0.04 mmol) and hexabutyl ditin (0.508 g, 0.875 mmol). The mixture was heated at 120° C. and stirred under argon for 16 h. The reaction solution was cooled to ambient temperature, filtered through CELITE to remove stannyl salts, and the solvent was removed by rotary evaporation. The resultant oil was purified by chromatography on silica gel (7.5 g) using 100% hexanes, followed by 20% ethyl acetate in hexanes, as the eluents. Fractions containing product were combined and evaporated to dryness to yield 7 as a pale yellow oil (0.200 g, 48% yield). ¹H NMR (400 MHz, CDCl₃) δ 9.00 (1H, d, J=1.2 Hz), 8.2 (1H, s), 7.53 (2H, d, J=7.2 Hz), 7.2-7.4 (5H, m), 6.9 (1H, t, J=4.4 Hz), 5.42 (2H, s), 1.52-1.62 (6H, m), 1.3-1.4 (7H, m), 1.14-1.2 (5H, t, J=8.0 Hz), 0.84-0.94 (9H, t, J=7.2 Hz). LCMS found 525.97, [M+H]⁺.

### Example 7

### Synthesis of 8-(Benzyloxy)-3-iodoquinoline

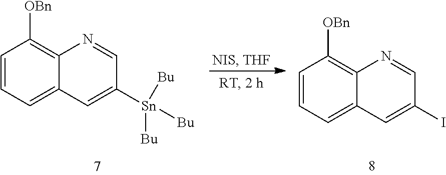

8-(Benzyloxy)-3-(tributylstannyl)quinoline (7) (0.257 g, 0.490 mmol) was dissolved in 5 mL of anhydrous THF. The solution was degassed with argon before the addition of N-iodosuccinimide (0.121 g, 0.539 mmol). The reaction solution was stirred at ambient temperature for 2 h, and the solvent was removed by rotary evaporation. The crude product was dissolved in dichloromethane and washed with saturated sodium bicarbonate (10 mL×3), followed by brine (10 mL×1). The organic phases were combined, dried over MgSO₄ (anhydrous), filtered, and evaporated to dryness. The residue was purified by chromatography on silica gel (9 g) using a 10%-30% ethyl acetate in hexanes elution gradient. The fractions containing the product were combined and evaporated to dryness to give 8 as a white solid (0.128 g, 72.5% yield). ¹H NMR (400 MHz, CDCl₃) δ 9.08 (1H, s), 8.51 (1H, s), 7.5 (2H, d, J=7.2 Hz), 7.2-7.4 (5H, m), 7.05 (1H, d, J=8.0 Hz), 5.43 (2H, s). LCMS found 361.83, [M+H]⁺.

### Example 8

### Synthesis of 3-Iodo-8-hydroxyquinoline

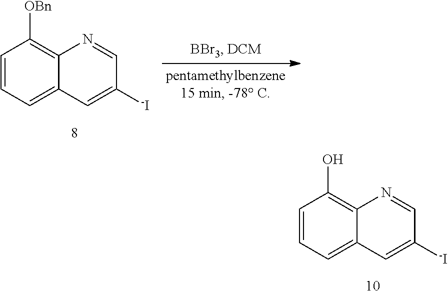

8-(Benzyloxy)-3-iodoquinoline (8) (100 mg, 0.318 mmol) was dissolved in 2.5 mL of anhydrous DCM. The solution was degassed with argon and stirred at −15° C. A solution of BBr₃ (1.0 M solution, 0.637 mmol) was injected drop wise into the solution over 10 min while the reaction mixture was cooled to −78° C. The solution was stirred at −78° C. for 30 minutes until the full conversion of 8 was determined by LC-MS. The reaction was quenched with the addition of 3 mL of a 10:1 chloroform/methanol solution. The crude solution was warmed to ambient temperature, evaporated to dryness, and purified by chromatography on silica gel (9 g) using a 100% dichloromethane to 10% methanol in dichloromethane elution gradient. The fractions containing the product were combined and evaporated to dryness to yield 10 as a white solid (0.069 g, 89% yield).

This application claims the priority of U.S. Provisional Application No. 62/161,473 filed 14 May 2015 and entitled “Preparation and Uses of Novel Quinolone Derivatives: Diagnostic and Therapeutic Agents for Alzheimer's Disease”, the whole of which is hereby incorporated by reference.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1. Alzheimer's Association Home Page. http://www.alz.org/(accessed Mar. 26, 2015).
2. Nasica-Labouze, J.; Nguyen, P. H.; Sterpone, F.; Berthoumieu, O.; Buchete, N.-V.; Coté, S.; De Simone, A.; Doig, A. J.; Peter Faller,‡ Garcia, A.; Alessandro Laio, A.; Li Mai, S. L.; Melchionna, S.; Mousseau, N.; Mu, Y.; Paravastu, A.; Pasquali, S.; Rosenman, D. J.; Strodel, B.; Tarus, B.; Viles, J. H.; Zhang, T.; Wang, C.; Derreumaux, P. Amyloid β protein and Alzheimer's Disease: When computer simulations complement experimental studies, J Am Chem Soc 2015, DOI: 10.1021/cr500638n.
3. Kung, Hank F. The β-Amyloid Hypothesis in Alzheimer's Disease: Seeing Is Believing, ACS Medicinal Chemistry Letters (2012), 3(4), 265-267.
4. Cohen, Ann D.; Rabinovici, Gil D.; Mathis, Chester A.; Jagust, William J.; Klunk, William E.; Ikonomovic, Milos D. Using Pittsburgh compound B for in vivo PET imaging of fibrillar amyloid-beta, Advances in Pharmacology (San Diego, Calif., United States) (2012), 64(Current State of Alzheimer's Disease Research and Theraputics), 27-81.
5. Melissa E. Murray, Val J. Lowe, Neill R. Graff-Radford, Amanda M. Liesinger, Ashley Cannon, Scott A. Przybelski, Bhupendra Rawal, Joseph E. Parisi, Ronald C. Petersen, Kejal Kantarci, Owen A. Ross, Ranjan Duara, David S. Knopman, Clifford R. Jack, Dennis W. Dickson, Clinicopathologic and 11C-Pittsburgh compound B implications of Thal amyloid phase across the Alzheimer's disease spectrum, Brain, 2015, Advance Access published Mar. 23, 2015: dx.doi.org/10.1093/brain/awv050.
6. Landau, S. M.; Thomas, B. A.; Thurfjell, L.; Schmidt, M.; Margolin, R.; Mintun, M.; Pontecorvo, M.; Baker, S. L.; Jagust, W. J, Amyloid PET imaging in Alzheimer's disease: a comparison of three radiotracers, European Journal of Nuclear Medicine and Molecular Imaging (2014), 41(7), 1398-1407.
7. Kobylecki, Christopher; Langheinrich, Tobias; Hinz, Rainer; Vardy, Emma R. L. C.; Brown, Gavin; Martino, Maria-Elena; Haense, Cathleen; Richardson, Anna M.; Gerhard, Alexander; Anton-Rodriguez, Jose M.; et al, 18F-florbetapir PET in patients with frontotemporal dementia and Alzheimer disease, Journal of Nuclear Medicine (2015), 56(3), 386-391.
8. Becker, Georg A.; Ichise, Masanori; Barthel, Henryk; Luthardt, Julia; Patt, Marianne; Seese, Anita; Schultze-Mosgau, Marcus; Rohde, Beate; Gertz, Hermann-Josef; Reininger, Cornelia; et al, PET quantification of 18F-florbetaben binding to β-amyloid deposits in human brains, Journal of Nuclear Medicine (2013), 54(5), 723-731.
9. Villemagne, Victor L.; Mulligan, Rachel S.; Pejoska, Svetlana; Ong, Kevin; Jones, Gareth; O'Keefe, Graeme; Chan, J. Gordon; Young, Kenneth; Tochon-Danguy, Henri; Masters, Colin L.; et al, Comparison of 11C-PiB and 18F-florbetaben for Aβ imaging in ageing and Alzheimer's disease, European Journal of Nuclear Medicine and Molecular Imaging (2012), 39(6), 983-989.
10. Curtis Craig; Gamez Jose E; Singh Upinder; Sadowsky Carl H; Villena Teresa; Sabbagh Marwan N; Beach Thomas G; Duara Ranjan; Fleisher Adam S; Frey Kirk A; et al, Phase 3 trial of flutemetamol labeled with radioactive fluorine 18 imaging and neuritic plaque density, JAMA neurology (2015), 72(3), 287-94.
11. Hatashita, Shizuo; Yamasaki, Hidetomo; Suzuki, Yutaka; Tanaka, Kumiko; Wakebe, Daichi; Hayakawa, Hideki, [18F]Flutemetamol amyloid-beta PET imaging compared with [11C]PIB across the spectrum of Alzheimer's disease, European Journal of Nuclear Medicine and Molecular Imaging (2014), 41(2), 290-300.
12. Zeng Fanxing; Goodman Mark M, Fluorine-18 radiolabeled heterocycles as PET tracers for imaging β-amyloid plaques in Alzheimer's disease, Current topics in medicinal chemistry (2013), 13(8), 909-19.
13. Ono, Masahiro; Saji, Hideo, Recent advances in molecular imaging probes for β-amyloid plaques, MedChemComm (2015), 6(3), 391-402.
14. Yang, Yanping; Cui, Mengchao, Radiolabeled bioactive benzoheterocycles for imaging β-amyloid plaques in Alzheimer's disease, European Journal of Medicinal Chemistry (2014), 87, 703-721.
15. Mach, Robert H, New targets for the development of PET tracers for imaging neurodegeneration in Alzheimer disease, Journal of Nuclear Medicine (2014), 55(8), 1221-1224.
16. Cui, Mengchao, Past and Recent Progress of Molecular Imaging Probes for β-Amyloid Plaques in the Brain, Current Medicinal Chemistry (2014), 21(1), 82-112.
17. Noel, Sabrina; Cadet, Sarah; Gras, Emmanuel; Hureau, Christelle, The benzazole scaffold: a SWAT to combat Alzheimer's disease, Chemical Society Reviews (2013), 42(19), 7747-7762.
18. Jason P. Holland,a,b Steven H. Liang,a Benjamin H. Rotstein,a Thomas L. Collier,a,c Nickeisha A. Stephenson,a Ivan Greguric,band Neil Vasdev, Alternative approaches for PET radiotracer development in Alzheimer's disease: imaging beyond plaque, Journal of Labelled Compounds and Radiopharmaceuticals (2014), 57(4), 323-331.
19. Ariza, Manuela; Kolb, Hartmuth C.; Moechars, Dieder; Rombouts, Frederik; Andres, Jose Ignacio, Tau Positron Emission Tomography (PET) Imaging: Past, Present, and Future, Journal of Medicinal Chemistry (2015).
20. Petric, Andrej; Johnson, Scott A.; Pham, Hung V.; Li, Ying; Ceh, Simon; Golobic, Amalija; Agdeppa, Eric D.; Timbol, Gerald; Liu, Jie; Keum, Gyochang; et al, Dicyanovinylnaphthalenes for neuroimaging of amyloids and relationships of electronic structures and geometries to binding affinities, Proceedings of the National Academy of Sciences of the United States of America (2012), 109(41), 16492-16497.
21. Tago, Tetsuro; Furumoto, Shozo; Okamura, Nobuyuki; Harada, Ryuichi; Ishikawa, Yoichi; Arai, Hiroyuki; Yanai, Kazuhiko; Iwata, Ren; Kudo, Yukitsuka, Synthesis and preliminary evaluation of 2-arylhydroxyquinoline derivatives for tau imaging, Journal of Labelled Compounds and Radiopharmaceuticals (2014), 57(1), 18-24.
22. Vasdev, Neil; Cao, Pengpeng; van Oosten, Erik M.; Wilson, Alan A.; Houle, Sylvain; Hao, Guiyang; Sun, Xiankai; Slavine, Nikolai; Alhasan, Mustafa; Antich, Peter P.; et al, Synthesis and PET imaging studies of [18F]2-fluoroquinolin-8-ol ([18F]CABS13) in transgenic mouse models of Alzheimer's disease, MedChemComm (2012), 3(10), 1228-1230.
23. Chien, David T.; Bahri, Shadfar; Szardenings, A. Katrin; Walsh, Joseph C.; Mu, Fanrong; Su, Min-Ying; Shankle, William R.; Elizarov, Arkadij; Kolb, Hartmuth C., Early Clinical PET Imaging Results with the Novel PHF-Tau Radioligand [F-18]-T807, Journal of Alzheimer's Disease (2013), 34(2), 457-468.
24. Matsumura, Kenji; Ono, Masahiro; Kimura, Hiroyuki; Ueda, Masashi; Nakamoto, Yuji; Togashi, Kaori; Okamoto, Yoko; Ihara, Masafumi; Takahashi, Ryosuke; Saji, Hideo, 18F-Labeled Phenyldiazenyl Benzothiazole for in Vivo Imaging of Neurofibrillary Tangles in Alzheimer's Disease Brains, ACS Medicinal Chemistry Letters (2012), 3(1), 58-62.
25. Liang, Steven H.; Holland, Jason P.; Stephenson, Nickeisha A.; Kassenbrock, Alina; Rotstein, Benjamin H.; Daignault, Cory P.; Lewis, Rebecca; Collier, Lee; Hooker, Jacob M.; Vasdev, Neil, PET Neuroimaging Studies of [18F]CABS13 in a Double Transgenic Mouse Model of Alzheimer's Disease and Nonhuman Primates, ACS Chemical Neuroscience (2015).
26. Valentina Oliveri,† Giuseppa I. Grasso,‡ Francesco Bellia,‡ Francesco Attanasio,‡ Maurizio Viale,§ and Graziella Vecchio, Soluble Sugar-Based Quinoline Derivatives as New Antioxidant Modulators of Metal-Induced Amyloid Aggregation, Inorg. Chem. 2015, 54, 2591-2602.
27. Fei Mao,a Jun Yan,a Jianheng Li,a Xian Jia,b Hui Miao,a Yang Sun,a Ling Huang*a and Xingshu Li, New multi-target-directed small molecules against Alzheimer's disease: a combination of resveratrol and clioquinol, Org. Biomol. Chem., 2014, 12, 5936-5944.
28. Yu'ning Song,†ac Hao Xu,†b Wenmin Chen,a Peng Zhan*a and Xinyong Liu, 8-Hydroxyquinoline: a privileged structure with a broad-ranging pharmacological potential, Med. Chem. Commun., 2015, 6, 61-74.
29. Liang, Steven H.; Collier, Thomas Lee; Rotstein, Benjamin H.; Lewis, Rebecca; Steck, Mia; Vasdev, Neil, Rapid microfluidic flow hydrogenation for reduction or deprotection of 18F-labeled compounds, Chemical Communications (Cambridge, United Kingdom) (2013), 49(78), 8755-8757.
30. Papazian, V.; Jackson, T.; Pham, T.; Liu, X.; Greguric, 1.; Loc'h, C.; Rowe, C.; Villemagne, V.; Masters, C. L.; Katsifis, A, The preparation of 123/1251-clioquinol for the study of Aβ protein in Alzheimer's disease, Journal of Labelled Compounds & Radiopharmaceuticals (2005), 48(7), 473-484.
31. Kulkarni, P. V.; Arora, V.; Roney, A. C.; White, C.; Bennett, M.; Antich, P. P.; Bonte, F. J. Radiolabeled probes for imaging Alzheimer's plaques, Nuclear Instruments & Methods in Physics Research, Section B: Beam Interactions with Materials and Atoms (2005), 241(1-4), 676-680.
32. Bader, Mamoun M.; Hamada, Tomoyuki; Kakuta, Atsushi, Theoretical investigation of the geometric structures and the second-order nonlinear optical properties of 8-hydroxyquinoline derivatives, Journal of the American Chemical Society (1992), 114(16), 6475-9.
33. Jung-Suk Choia, Joseph J. Braymerb, Ravi P. R. Nang, Ayyalusamy Ramamoorthy, and Mi Hee Lim, Design of small molecules that target metal-Aβ species and regulate metal-induced Aβ aggregation and neurotoxicity, PNAS, 2010, 107, 21990-21995.
34. Scott, L. E.; Orvig, C. Medicinal inorganic chemistry approaches to passivation and removal of aberrant metal ions in disease, Chem Rev, 2009, 109, 4885-4910.

That which is claimed is:

1. A compound having the formula: 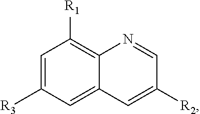
wherein R₁ is selected from the group consisting of OH, OCH₂C₆H₅, NO₂, NH₂, aminoacyl, aminosulfonyl, NHCH₃, and N(CH₃)₂; and R₂ and R₃ are independently selected from the group consisting of F, Cl, Br, I, or aryl, heteroaryl, CH═CH-aryl, or CH═CH-heteroaryl, wherein any of the aforementioned aryl groups can be halo-substituted at any position.

2. The compound of claim 1, wherein at least one of R and R₃ is selected from radioisotopes of F, Br, and I.

3. The compound of claim 2, wherein at least one of R₂ and R₃ is ¹⁸F or ¹²³I.

4. The compound of claim 1 that binds to amyloid plaques and/or neurofibrillary tangles in a mammal.

5. The compound of claim 4, wherein the amyloid plaques comprise beta amyloid (Aβ) and/or the neurofibrillary tangles comprise tau.

6. The compound of claim 1 that prevents and/or reverses the formation of amyloid plaques and/or neurofibrillary tangles in a mammal.

7. The compound of claim 1 that binds a metal ion.

8. The compound of claim 7, wherein the metal ion is an ion of iron, copper, or zinc.

9. The compound of claim 1 that is selected from the following compounds, wherein X is a halogen selected from F, Br, and I, and wherein R is H or C1-C6 alkyl:

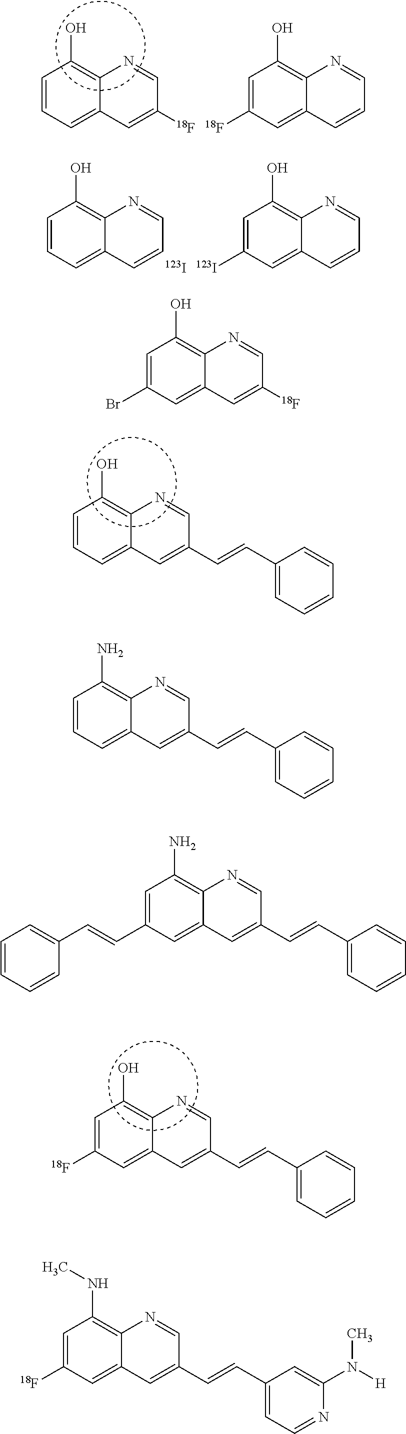

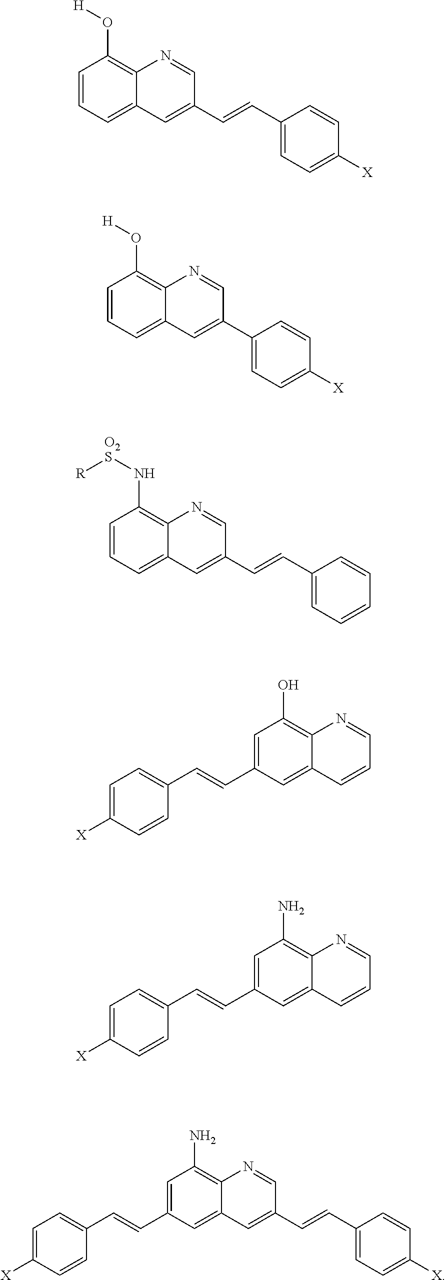

10. The compound of claim 1 that is capable of crossing the blood-brain barrier of a mammal.

11. The compound of claim 1 that is an imaging agent for visualizing amyloid beta plaques and/or tau neurofibrillary tangles in the brain of a mammal.

12. A pharmaceutical composition comprising the compound of claim 1 and an excipient, carrier, or diluent.

13. A method of diagnosing or prognosing a disease or condition in a subject, the disease or condition associated with a presence of amyloid deposits and/or neurofibrillary tangles in an organ or tissue of the subject, the method comprising the step of administering a compound of claim 11 to the subject, whereby the presence of amyloid deposits and/or neurofibrillary tangles is detected.

14. The method of claim 13, wherein the disease or condition is associated with the presence of amyloid deposits, and wherein the disease or condition is selected from the group consisting of Alzheimer's disease, Parkinson's disease, Huntington's disease, spongiform encephalopathy, diabetes mellitus type 2, fatal familial insomnia, atrial amyloidosis, atherosclerosis, rheumatoid arthritis, familial amyloid polyneuropathy, hereditary non-neuropathic systemic amyloidosis, dialysis related amyloidosis, cerebral amyloid angiopathy, and systemic AL amyloidosis.

15. The method of claim 13, wherein the disease or condition is associated with the presence of neurofibrillary tangles, and wherein the disease or condition is selected from the group consisting of Alzheimer's disease, progressive supranuclear palsy, chronic traumatic encephalopathy, primary age-related tauopathy, corticobasal degeneration, and postencephalitic parkinsonism.

16. A method to aid in treating or preventing a disease or condition in a subject, the disease or condition associated with a presence of amyloid deposits and/or neurofibrillary tangles in the subject, the method comprising the step of administering a compound of claim 1 to the subject, whereby amyloid deposits and/or neurofibrillary tangles in the subject are decreased.

17. The method of claim 16, wherein the disease or condition is associated with the presence of amyloid deposits, and wherein the disease or condition is selected from the group consisting of Alzheimer's disease, Parkinson's disease, Huntington's disease, spongiform encephalopathy, diabetes mellitus type 2, fatal familial insomnia, atrial amyloidosis, atherosclerosis, rheumatoid arthritis, familial amyloid polyneuropathy, hereditary non-neuropathic systemic amyloidosis, dialysis related amyloidosis, cerebral amyloid angiopathy, and systemic AL amyloidosis.

18. The method of claim 16, wherein the disease or condition is associated with the presence of neurofibrillary tangles, and wherein the disease or condition is selected from the group consisting of Alzheimer's disease, progressive supranuclear palsy, chronic traumatic encephalopathy, primary age-related tauopathy, corticobasal degeneration, and postencephalitic parkinsonism.

19. A method of synthesizing a compound of claim 1, the method comprising the steps of:
(a) preparing an intermediate compound which is 8-(benzyloxy)-3-(tributylstannyl)quinoline or 8-(benzyloxy)-6-(tributylstannyl)quinoline;
(b) subjecting the intermediate to halodestannylation to obtain 8-(benzyloxy)-3-haloquinoline or 8-(benzyloxy)-6-haloquinoline; and
(c) subjecting the product obtained in (b) to debenzylation to obtain 3-halo-8-hydroxyquinoline or 6-halo-8-hydroxyquinoline;
wherein halo represents F, Br, or I.

20. The method of claim 19, wherein step (b) comprises radiohalodestannylation, and in step (c) 3-radiohalo-8-hydroxyquinoline or 6-radiohalo-8-hydroxyquinoline is obtained.
